(19) United States
(12) Patent Application Publication
(10) Pub. No.: US 2009/0093761 A1
(43) Pub. Date: Apr. 9, 2009

(54) Medical-procedure assistance device and method with improved optical contrast, and new practitioner-safety, device-fixation, electrode and magnetic treatment and lumen-dilation capabilities

(76) Inventors: John W. Sliwa, Los Altos, CA (US); Bryan T. Oronsky, Los Altos, CA (US); Herbert L. Berman, Los Altos Hills, CA (US); Neil C. Oronsky, Los Altos Hills, CA (US); Carol A Tosaya, Los Altos Hills, CA (US); George W. Keilman, Woodinville, WA (US)

Correspondence Address: David W.Collins;Intellectual Property Law, Suite 100, 512 E.Whitehouse Canyon Road, Green Valley, AZ, 85614, US

(21) Appl. No.: 11/973,052

(22) Filed: Oct. 5, 2007

(51) Int. Cl. A61M 5/32

(52) U.S. Cl. 604/116

(57) ABSTRACT

Many medical procedures, such as needle-sticking, could benefit from an assistive device that improves the optical contrast of externally targeted features and lumens of interest residing in and underneath the skin and/or exposed organ tissues. The inventive inexpensive device and method are useable on such externally targeted features and lumens while also protecting the practitioner and freeing up both of his/her hands, if necessary, to thereby eliminate practitioner self-sticking problems. The present device provides good optical contrast and also provides splash-protection against HIV, hepatitis and other blood-borne diseases. The inventive device method and apparatus may also include vibratory subcutaneous electrical nerve stimulation (TENS), drug-based or heating treatment capabilities for reducing pain, both perceived and real pain, associated with a device guided procedure. Finally, the pain reduction mechanisms have also been found useful for lumen dilation.

### BACKGROUND ART

There are pocket-transportable inexpensive medical optical assistive devices (e.g., $200-$400 cost) and one very exceedingly expensive floor-mounted ($25,000 cost) medical optical assistive device of this general sort in the prior art of guiding devices for the insertion of needles into veins, for example, to draw blood or to give an injection (“needle-sticking”). Needle-sticking is a particularly exacerbating problem with older people, whose veins are notoriously difficult to find and to position.

The inexpensive devices, of which the Veinlite EMS® viewable at www.veinlite.com, Sugar Land, Tex. is a typical representative example, all give relatively poor optical contrast of subsurface lumens for their stated purpose of needle-stick guidance or subsurface lumen inspection. Further, they do not protect the practitioner from blood (or bodily fluid) splashes and they require the practitioner to utilize one hand to hold the device while the other hand does the sticking. This juggling invites practitioner self-inflicted needle-sticks which cause considerable mental anguish for the practitioner when accidentally stuck as he/she frequently is obliged to receive preventative treatments against blood-borne diseases which the patient might (or might not) have.

The prior art devices also offer no features or capabilities to address pain or to deliver any type of patient-beneficial therapy or treatment, such as for the potential or perceived pain caused by carrying out a needle or puncture-related procedure such as a needle-stick or needle-biopsy. Our invention herein addresses all of these issues by providing one or more cooperative means of improved device optical contrast, a means of device skin or tissue/limb attachment, juxtaposition or presentation and a means of blood-splash protection for the practitioner, all preferably in a lab coat pocketable device. We also provide multiple means for pain reduction and several means of enhancing lumen dilation such as for making a needle-stick easier. We further extend our device's novel integrated therapy capabilities beyond just pain treatment by making it capable of delivering electrode-excitation based therapeutic treatments and magnetic treatments of a variety of types—not just pain reduction therapies. Finally, we also enable our device to provide some physical guidance or mounting for procedural implements such as blood-sampling and biopsy needles. Thus the device can guide the practitioner to tissue targets of interest as well as help him/her guide a procedural implement to if not also into the tissue. The implement guiding can be both visual in nature (seeing the needle and/or target-possibly including while inside the tissue) as well as physical (helping the practitioner align or hold a procedural implement).

The very expensive VeinViewer® device from Luminetx of Memphis, Tenn. viewable at www.luminetx.com is not the sort of device that a doctor or practitioner could ever carry around in his/her pocket even if he/she desired to do so. That device utilizes infrared subsurface tissue imaging and reprojection of the imaged infrared contrast as human-visible optical contrast upon the overlying superficial skin surface. At $25,000 per system, still only one patient can be treated at a time. From our own developmental efforts, we do not see this expensive unit providing proportionally better procedure assistance than the far more inexpensive prior art devices nor than our inexpensive inventive device. In essence, the inexpensive units are about a hundred times more cost efficient-particularly ours herein which provides superior optical contrast and is safer and easier to use and optionally offers a variety of pain-control and electrode-based or magnetic-based therapy capabilities never seen before in such an assistive/guidance device.

### SUMMARY OF THE INVENTION

We provide an improved device that solves the optical-contrast problem, the blood splash problem, and allows for the practitioner to utilize both hands (as needed) to undertake his/her medical procedure or intervention without having to hold or manipulate the assistive device with one hand while doing so if he/she so desires. This will reduce or completely avoid self-inflicted practitioner needle-sticks, as the practitioner will not be juggling two separate apparatus (e.g., assistive device and syringe) at the same time.

We further provide a means to manipulate blood flow in the limb as an integral part of the device solution, if that is also desired. Also new is the delivery of pain-management and other electrotherapies or magnetic-based therapies from such an optically assistive/guidance device. Such pain may or may not be related to the intended action of needle sticking or tissue penetration. The pain-therapy or other deliverable therapy may be related to a preexisting problem in the patients body (e.g., varicose veins or a muscle injury for example) and the assistive device assists in delivering the therapy to that location. Thus some applications of the device will not involve needle-sticking or skin-puncturing and may not even involve pain. The preferred device is pocketable, can run on a rechargeable lithium-ion or nickel-metal hydride battery, or can run off of an external battery charger or power pack. The optional device capabilities in a product would likely include the electrotherapy and magnetic therapy features and lumen-dilation features.

We anticipate device use in both an examination-only mode and an examination plus procedure and/or therapy mode. One may also choose to deliver only a therapy. Therefore, the device can also be considered a combination device whose one or more capabilities may be utilized on a given patient.

### BRIEF DESCRIPTION OF THE DRAWINGS

FIG. 1 depicts our skin or limb-conforming assistive device in four standard orthogonal views. Specifically, FIG. 1A is an end view of the device, FIG. 1B is a side view of the device, FIG. 1C is a bottom (skin-side) view of the device, and FIG. 1D is a top view (practitioner-observed side) of the device.

FIG. 2 depicts the same device in a perspective 3-D view in which we also show device mounting straps, an optical filter and/or splash guard and an internal rechargeable battery. An optically-contrasted portion of a subsurface lumen feature of interest can also be seen.

### DETAILED DESCRIPTION OF THE INVENTION

### A. Terminology

Colors, in terms of wavelength ranges, overlap to a degree as does human color judgment:

| | |
|---|---|
| Yellow | ca 570-600 nanometers |
| Amber | ca 588-610 nanometers |
| Orange | ca 590-635 nanometers |
| Red | ca 620-750 nanometers |
| White | ca 400-800 nanometers |
| NIR or Near Infrared | ca 0.7-1.0 microns |
| SWIR or Short Wave Infrared | ca 1.0-3.0 microns |
| MWIR (or MIR) or Mid Infrared | ca 3.0-8.0 microns |
| LWIR (or LIR) or Long Wave Infrared | ca 8.0-12.0 microns |
| VLWIR or Very Long Wave Infrared | ca 12-30 microns |
| UV or Ultraviolet | ca 255-420 nanometers. |

By “yellow-orange” or “orange-yellow” we mean containing at least one wavelength from each of the above orange and yellow wavelength ranges.

By “orange-red” or “red-orange” we mean containing at least one wavelength from each of the above red and orange wavelength ranges.

By “white” we mean having mixed wavelength content generally across or distributed continuously or discretely across some or most of the white wavelength range stated above.

By “infrared” we mean at least one wavelength of the NIR, SWIR, MWIR, LWIR or VLWIR ranges unless the specific range, (e.g., NIR) is specified. This will typically be a wavelength longer than 0.7 microns.

Electrotherapies and Electrode-Based Therapies
a) TENS: (Transcutaneous Electrical Nerve Stimulation). TENS units typically allow the user to select high-frequency stimulation (60-200 Hz) or low-frequency stimulation (<10 Hz). We have provided several sources for such existing products below. The below units typically can deliver other electrotherapies as well such as NMES below. It is the integration of the electrotherapy (and/or magnetic therapy) capability in our assistive/guidance optical-contrast device and method which we consider novel.
b) IFC (Interferential Current) is a deeper form of TENS, typically modulating a high frequency (4000 Hz) carrier waveform with the same signal produced by a TENS unit. The high frequency carrier waveform penetrates the skin more deeply than a regular TENS unit, with less user discomfort for a given level of stimulation.
c) NMES (Neuromuscular Electronic Stimulation) typically is around 750 Hz. Muscles are stimulated through the skin.
d) GS (Galvanic Stimulation) also known as galvanic revivification, involves the discharge of a battery into the body which is delivered, transcutaneously, to the site of the neuromuscular junction, also called the motor end plate region. While open circuit voltages can be as high as 100 volts DC, the current passed should not exceed 5 to 50 milliamperes, and should be delivered in pulsed form, in simulation of the way the nervous system delivers energy to post synaptic structures, be they organs or muscles. These pulses can be as brief as ¼ of a millisecond, and the frequency of pulses can be as high as 900 Hz. This transcutaneous delivery can be achieved using a wand, or merely immersion into water containing one of the electrodes, while the other is attached to the body outside of the water. The pulse delivered to the motor endplate region comes from the anode, not the cathode. The cathode should be allowed to corrode so that the electrical charge delivered to the body is greater than the electrical charge removed from the body by the cathode.
e) Other forms of electrotherapy include Scenar, Ultrasound-based (US), Pulsed Shortwave Diathermy (PSWD), microcurrent, Russian-Hi Volt, Interferential Therapy (I/F), TECAR therapy, Laser Therapy and Combination therapies from the corroding cathode and the battery.
f) Electrophoresis (an Electrode Based Therapy): The driving of charged entities inwards or outwards-such as drugs or ions, through a medium utilizing electrical fields
g) Iontophoresis (an Electrode Based Therapy): This is similar to electrophoresis but usually involving the driving of ionic species such as drugs or body fluid constituents into (or out on tissues.

Examples of TENS and other electrotherapy device sources include:
(i) Empi, St Paul, Minn., Select™ (TEMS) Pain-Management System, at www.empi.com;
(ii) Medical Products Online Inc, distributor of the ReliaMed 350 TENS unit at www.medicalproductsonline.com; and
(iii) Active Forever.com, distributor of the ReliaMed GM320T TENS unit at www.activeforever.com;
(iv) ReliaMed itself is in Fort Worth, Tex. at www.reliamedproducts.com.
Other of its TEMS models include the digital ZZA320C, the analog ZZA350T, and the analog ZZA350.
(v) RS Medical of Vancouver, Wash., (www.rsmedical.com), Physician-prescribed electrotherapy rehabilitation products.

Many such electrotherapy units utilize carbon electrodes with a stickable gel; however, it is possible to utilize needle or acupuncture needles as electrodes as well. We include in our inventive scope the use of a procedural needle or other puncturing or cutting implement as a TENS electrode (or as an electrode for any other inventive electrical or electrotherapy therapy). Our device housing might also be or support (or comprise) a TENS or other electrotherapy electrode(s) such as on its skin-contacting surface.
h) Magnetic Therapies
   Modern electromagnetic and magnetic therapies (similarly called biomagnetics, electrobiomagnetics, magnet therapy, magnetic energy therapy, magnetic field therapy, magnetic stimulation, and magnetotherapy) are the use of magnetic fields, magnets, and magnetic devices to treat various physical and emotional conditions. These therapies are based on the premise that electrical activity exists in the body at all times, particularly as the heart beats or during bone production. Healing of various injuries or the damage done by some diseases is also proposed to be enabled or accelerated with the help of applied magnetic fields. Magnetic fields have been combined with warming infrared light-something possible with our inventive device. Magnetic therapies will utilize non-invasive or invasive permanent magnets and/or electromagnets. Thus, our device and/or a procedural implement used therewith may apply one or more static or time-varying magnetic fields.
   A large selection of online vendors with a broad variety of magnetic field applying devices exist, some of which are listed below:
   www.magnetemporium.com;
   www.bioflexmagnets.com;
   www.magnetsandhealth.com;
   www.therionresearch.com; and
   www.e-magnetshop.com.

### B. Discussion of the Preferred Embodiments

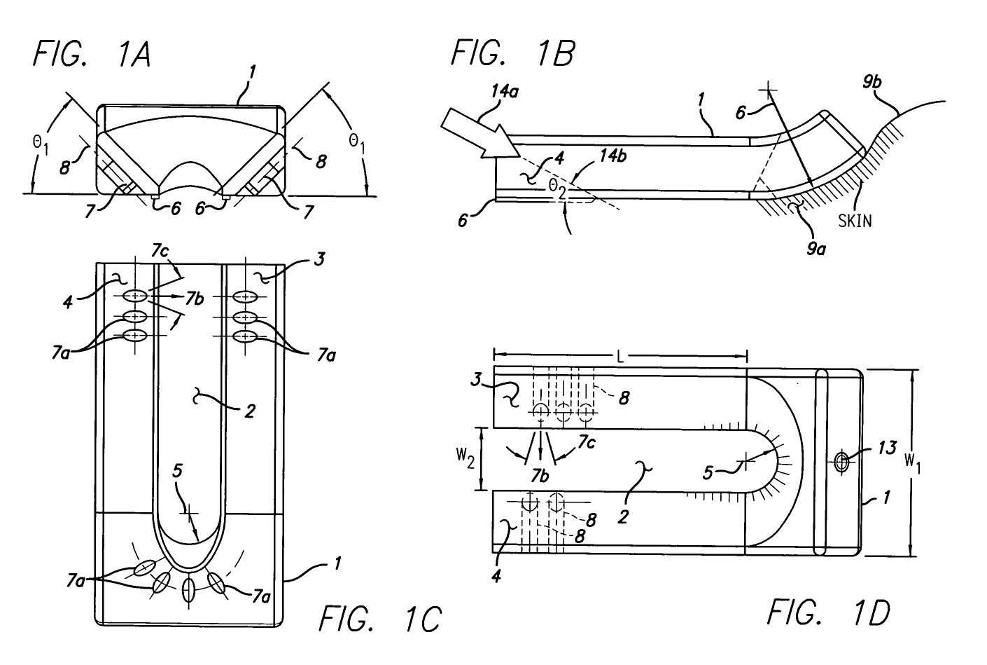

Let us begin with FIG. 1. Therein we see an assistive/guiding device in four orthogonal views (FIGS. 1A-1D). The inventive device 1, which primarily consists of a housing, defines a viewable tissue region 2 which will provide superior optically-contrasted views of lumens and subsurface features (and possibly of the procedural implement inside the tissue) thereunder. We shall refer to this practitioner-observable optically-contrasted region 2 as the “second tissue portion” as opposed to the “first tissue portion” to be discussed below whereat the light causing the useful optical contrast in region 2 is delivered into the tissues. The upper right side-view (FIG. 1B) depicts the device 1 juxtaposed to a patient's skin 9a/9b during use.

The particular example device 1 shown in FIGS. 1A-1D has a general U-shape or slot shape with extended arms 3 and 4 that define optically-contrasted tissue region 2. Note that the optical-contrast region 2 is generally slot-shaped as depicted in this particular example and is of general width W2 and length L. Further note that the slot-end has a radius 5. The tissue region 2 is often referred to as slot 2 herein as it has an extended shape. Thus the entire viewable contrast region 2 comprises the rectangular slot plus its additional radiused end in this depiction.

Referring now to FIG. 1B, we see the device 1 juxtaposed to a patient's arm skin 9a/9b. In this example, the patient's skin region 9b is the crook of the elbow (cubital fossa or anterior cubital fossa) while region 9a is the adjacent forearm. Many needle-sticks into the basilic, cephalic and/or median cubital vein take place at or near this location. Note that device 1 has a bottom-surface radius 5 which conformably fits the crook of the elbow while the flatter arm regions 3 and 4 conformably fit the forearm regions of the patient's tissues. A repeated theme of this teaching will be that we desire to inject light into tissue through a first tissue portion in a manner wherein it produces useful optical contrast at a second tissue portion, without unacceptable or interfering light leakage across the external tissue surface. Thus, skin or tissue-conformance to our device is one way to prevent such lateral light surface leakage which would decrease the desired optical contrast by optically washing it out. A compressable light-gasket makes such conformance easier. A second additional or alternative approach to controlling surface-lateral light leakage is to simply make sure that little or no light is directed out into the region 2 on the surface-skin (as opposed to injected beneath it). This can be accomplished, even if the device is held above the skin with somewhat of a gap, by directing the light sources steeply downward and/or keeping their divergence angles small.

Most often, but not always, the second tissue region whereat optical contrast is viewed will be a separate region such as the slot 2/radius 5 region while the light causing the optical contrast will be injected at a first tissue region, separate from the second region. In the FIG. 1 case, the light injection regions or first tissue portions are primarily under the arms 3 and 4 and radius 5, the arms and radius also primarily defining slot 2. So the arms 3 and 4 and radius 5 are injecting the light in a first tissue subportion(s) and causing the desired optical contrast in the second tissue subportion 2/radius 5. Note that light injection is also implemented in this example device 1 along the radius 5 slot-end region as seen in underside view 1c.

Continuing with FIG. 1, we see that the skin-adjacent bottom-surface of device 1 (FIG. 1C) has a light-gasket, lip or ridge 6 which helps to prevent injected light from laterally surface-lighting the tissue. Illumination light sources 7 are mounted in holes 8 in a manner such that they can inject light into tissues 9a/9b for example. Note that in the FIG. 1 example, the multiplicity of light locations 8 are arranged around slot 2 and radiused slot-end 5 such that they substantially inject their light into the subsurface of tissue viewable inside slot 2 and radius 5. In other words, they provide light with which lumens or other features in or under the slot 2/radius 5 tissue region can be optically-contrasted as by backlighting, by light up-scattered from below, or optically-contrasted by light impinging from above or from the sides of such lumens or features of interest.

The example light sources 7 are mounted in holes 8 that intersect the bottom (skin) surface of the device with elliptical apertures 7a. This shape is the natural result of a cylindrical hole 8 intersecting a plane at an angle to the hole axis.

It will be noted in FIG. 1 that the viewable-contrast slot region 2 perimeter, as defined by arms 3 and 4 and radius 5, is tapered at an angle theta 1 of about 45 degrees looking at end-view FIG. 1a. This taper allows easy viewing (e.g. as seen in FIG. 1D and at non-normal observation angles not-shown) of the optical contrast being created in slot region 2/radius 5 from above and from an angle to the side and further, allows for a medical implement such as a syringe-needle to be delivered to the observed target such as along the depicted approach path 14a at an angle theta 2 shown in FIG. 1B. Note that the procedural needle or syringe implement (not shown, and if any is used) approach path 14a is at an angle θ₂ (item 14b) with the surface skin. It will be appreciated from FIG. 1 that an implement-approach to the optically-contrasted target seen in slot 2/radius 5 allows for plenty of manipulative clearance for the needle (or other implement benefiting from the assistive/guiding device). The open-ended slot of width W2 at the mouth also offers an easy approach for a procedural implement (implement itself not shown). We emphasize that viewable second tissue portion 2/radius 5 may have other desirable fixed, changeable or adjustable shapes such as having curved arms 3, 4, or closed or connecting arms or shapeable arms. Such shaping may be in any number of dimensions including in three dimensions (fixed two dimensional generally rectangular shape shown within length L in the FIG. 1 depictions).

We earlier noted access and viewing enhancement taper angles theta 1 on the slot arms 3 and 4 and radius 5 and these are specifically depicted in FIG. 1 as having an angle θ₁ of about 45 degrees. Note that this taper, as shown, passes all the way around the radiused end of the slot as well. In this manner, the practitioner does not have to be directly above slot 2 (as he/she would be in FIG. 1D) to view or interact with tissue region 2 or with the guided procedural implement, if any is being employed. The depicted tapering may take any form and is not restricted to a fixed angle or even a flat faceted surface. Note that in FIG. 1 we show the individual light sources 7 as being conventional 3 or 5 mm diameter LEDs, preferably super-bright LEDs. They are shown mounted in holes 8 also shown at about 45 degrees to the skin surface 9a/9b such that they inject light underneath and into observable contrast region 2 and 5. The overall device is shown as having a width W1 and its overall length is somewhat longer than the arm(s) length L. A power-adapter or recharger electrical plug or jack is shown on the device's end as item 13. The adapter or recharger might also power the device without an internal battery, and if desired, may even power the device all the time such as in the case wherein the device has no battery at all or no battery available. The optional TENS or other electrotherapy or magnetic pain-reduction or other therapeutic-purpose feature to be discussed below in detail may also conveniently utilize power from the device housing such as from an additional jack (not shown) to which TENS or other electrodes or even electromagnets can be plugged into the housing 1. In that manner, the device 1 battery or power source preferably (but not necessarily) powers all power-requiring device features and capabilities used in or with the assistive/guiding device.

Note that each LED 7 in FIG. 1 emits light along a central axis 7b, which in the shown case intercepts the surface tissue at about 45 degrees nominal. Each light source 7 that is generally aligned to emit along a central or nominal beam path 7b may also be arranged to have a source divergence or spread-angle depicted by included angle 7c. So it will be appreciated that light is emitted in the depicted example generally aligned along beam paths 7b in the form of cone-shaped beams of included angle 7c in the depicted case of the sources having a rotationally symmetric divergence angle 7c. Note that if one were to choose the light 7 nominal angle or axis 7b at 45 degrees (as approximately shown) and a total included divergence angle of 90 degrees, then one would be emitting a 90 degree included-angle cone of light aligned along central beam line 7b with a 90 degree divergence angle 7c such that the cone of light (at its extreme extents) is just tangent to the skin line at the top (skin surface side) and is normal to the skin line (skin surface) on the far underside. In general, we wish not to inject too much light tangent to or upward from the skin, as it may be immediately leaked with little or no skin penetration depending on tissue shape and distortion. Some skin-injected or surface-lighting amount may be preferred, particularly when looking for in-skin or shallow features and tissue distortion or extrusion allows for such tangential or even upward-lighting. Of course, this simple calculation does not take into account that the tissue or skin surface is somewhat distorted or extruded by the device 1, depending on skin compliance and device loading such that the theoretically-flat tissue tangent-limit is not a rigid fixed limit and one might even upwards-light into an extruded tissue as mentioned.

Note that the light sources 7 arranged on the side arms 3 and 4 and radius 5 are depicted as generally having their central emission axes 7b approximately parallel to each other (at least on the arms 3, 4) given the straight-arm defined slot 2. This parallelism is not a requirement nor is the depicted straight inside slot-edges as mentioned. We have found that for a variety of shaped slots 2, such as for a generally rectangular/straight slot (shown) or circular, elliptical or parabolic slots or windows 2 (not shown), it is beneficial to have at least some of the light source 7 central beam alignment axes 7b be oriented approximately normal to the local slot edge (as shown for all of the light sources 7 in this example). Note that because the light sources 7 are closely packed and their central beam lines 7b may be parallel (as shown on the straight interior slot edges), because of their divergence cone angles 7c light from adjacent neighboring sources 7 overlaps and it overlaps coming from different directions (from differently situated light sources 7). Note also that light sources 7 mounted at the radiused end 5 of the slot 2 are each depicted arranged being approximately individually perpendicular to the local round slot edge, thereby causing the light sources alignment axes 7b to be at an angle to each other (not parallel like on the arms 3 and 4) in this radiused region. The present inventors have found that good optical contrast for a region such as slot 2 or radius 5 can be derived from one or both of a) close juxtaposition of generally edge-normal light sources 7 as on the straight interior slot edges), and/or b) close juxtaposition of nonparallel but locally radius-edge normal sources 7 as shown in the radius 5 region. This is not to say that straight edge non-perpendicular sources are unattractive; in fact, we have found them to be also useful.

We mentioned the use of a light dam or gasket such as item 6. We wish to again note that if the illumination sources 7 are sufficiently tilted upwards (towards being skin-normal (e.g., 45 to 90 degrees to skin) and have sufficiently narrow divergence angles that one may eliminate the light gasket 6 whose job is to prevent direct lighting of the skin surface. Such a device would be functional even off the skin surface with a gap between the device and the skin. The present inventors have also noted, particularly for light sources 7 approaching skin-normal light entry, that the entire device can be lifted off the skin surface somewhat and still function. In fact, we have noticed that by slightly lifting the device from the skin surface, we eliminate skin extrusion out of region 2. Such skin/tissue mounding or extrusion may cause shadowing of some or all of the extruded skin regions. By slightly lifting (or spacing) the device off the surface, we eliminate all such shadowing because the tissue is not extruded or mounded-up above the light beams by the device. Such “lifting” or standoff-presenting of the device can be accomplished by an underside spacer(s) (not shown) similar to that of item 6 but larger and more likely placed around the outboard edge of the device as opposed to the inboard edge where light gasket 6 is shown. Conveniently, this lifting spacer can be part of the device housing or can be a separate attached spacer(s).

Before discussing light sources, we wish to emphasize that the inventive device may take many physical shapes/forms beyond that shown in FIG. 1. Another particularly attractive form (not shown) is what we refer to as the “cuff” design. This device looks like a bracelet or arm-band which wraps around the arm, leg, ankle, calf or other limb. The wrapping direction is around the perimeter of the arm and may be partially wrapped or fully wrapped. Like an armband or wristband, if the wrapping closure is complete enough, then the device can self-clamp to the limb as by elastic or plastic deformation. If the device itself only wraps around a portion of the limb perimeter, then an attached strap or other fixation means may be used to hold it onto the limb. In one attractive design (not shown), such a cuff device would have an observable contrast region 2 or window which is elongated and also wrapped around the limb. Thus, the cuff band device would have an elongated window whose long dimension also wraps around a portion of the limb perimeter.

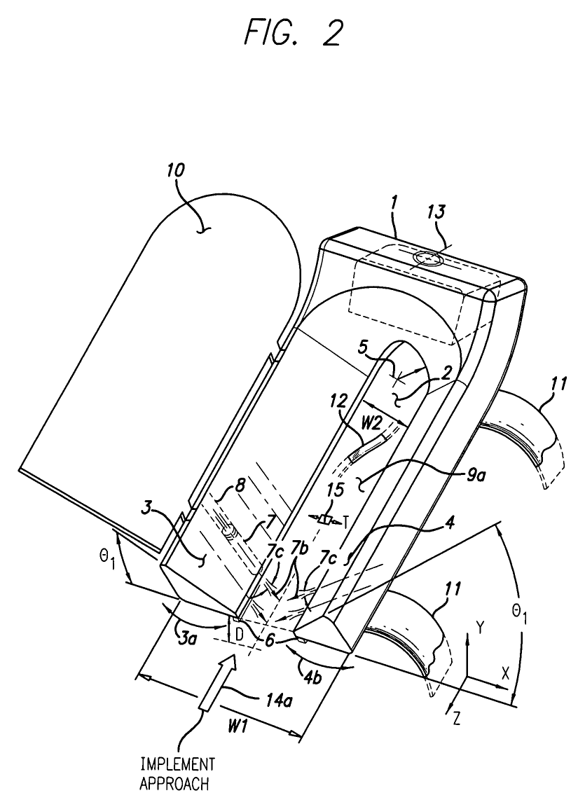

The above cuff-band device with an elongated co-wrapping observable window region 2 also does a particularly nice job of illuminating veins. For light sources situated on the long edges of the wrapping observable window, their light is passed into tissue generally parallel to the veins that mostly run up and down the arm. Because of this parallel illumination, one avoids circumferential shadowing of the veins themselves. In a preferred cuff-like device, it would wrap and self clamp on the arm and its elongated window would also peripherally or circumferentially wrap around some of the arm and have radiused or rounded ends. Light sources could be employed at the long edges as well as at the radiused ends. Similar to FIGS. 1 and 2, one would likely provide an access slot or depression to allow needle or implement delivery.

The present inventors' best design to date is a cuff-like design wherein the elongated cuff window portion containing the light array is relatively flat at the skinline whereas the remainder of the cuff is curved. We also found that we could populate that elongated relatively flat window only on its opposed long sides and did not need lights of the radiused ends. So this is a little like a large watch wherein the skin-contacting watch body is flat but the flexible band is curved and conforming. The lights in this array are directed in planes approximately parallel to the veins running up and down the legs and arms.

This is an appropriate point at which to discuss light source technology useable in the inventive device. To begin with, our elliptical (in this example) light apertures or windows 7a out of which light emanates into the skin or tissues 9a/9b may or may not contain a local bulb or LED light source. Indeed, in FIG. 1, we have shown each hole 8 as having a resident cylindrical super-bright LED. However, one could just as well have an optical fiber (not shown) deliver light to one or more emission apertures 7a; thus, from a technical point of view, the light source feeding that fiber can be physically remote from the emission aperture(s) 7a itself. So, to be all-inclusive, the apertures 7a emit light, which may or may not be locally produced by one or more local discrete or distributed illumination sources.

In general, we anticipate several different kinds of light sources useable in the inventive device. These include the following:
1) Discrete single-element light sources: A single-chip or single filament source is employed as for the depicted FIG. 1 example wherein we see a multiplicity of individual LEDs, each containing one LED chip, typically of one color and emission divergence angle. Each such source requires its own electrical connections (individually connected in series or parallel, for example). Often, many types of light sources useable in the inventive device have integrated or juxtaposable lenses or diffusers which can also define or modify divergence angles. Divergence patterns do not have to be cone-shaped; however, such sources are widely available. By “discrete” is meant individually powered or connected with wires or traces and/or individually packaged.
2) Discrete multielement light sources: Such sources may comprise a light source having a single package or substrate but having multiple internal or onboard light chips or filaments. Examples include ganged-multichip or multichip-multicolor LEDs. The package or surface-mount entity typically has shared electrical connections that power and/or switch all the contained chips/filaments. Frequently, multiple subchips are ganged in a single device like this to increase intensity, modify beam shape or to adjust color, assuming the subchips have different colors. These usually share one or more connecting pins on the package/substrate.
3) Distributed light sources: Unlike the above sources, these sources may any one or more of (a) utilize light-piping or light-distributing elements coupled to a localized bulb(s) or source(s), or (b) comprise 1-D or 2-D arrays of single-element or multielement discrete light sources of the above type 1/2, or (c) comprise 1-D or 2-D strips or panels of light emitting material such as OLED polymer films. The emitting area of any distributed source may be one-dimensional (e.g., straight, curvilinear), two-dimensional or even three-dimensional. In essence, distributed sources are usually needed if an extended lateral dimension or area is to be illuminated. This is frequently the case and our example figures herein will show devices with a distributed light source having a curvilinear shape comprised of multiple discrete light sources arranged in a 1-D row or array.

The present inventors have utilized red, red-orange, orange, orange-yellow, amber and yellow as well as white light sources and infrared light sources to good effect. Wavelengths in the red-red/orange-orange range or white give the best performance in a variety of skin and tissue types. Thus, inventive devices may be arranged, for example, to have any of a) only one wavelength or source color (e.g., all red or orange or white sources) or b) two or more different wavelength or color source types (e.g., some red sources and some orange or white sources), c) one or more source or color types having a distributed-wavelength or multiwavelength output capability (e.g., white or red-red/orange-orange), or d) one or more source or color types such as from light source (2) above wherein the light source can be operated at two or more different wavelengths or colors separately or simultaneously in time.

Further, we note that the inventive device 1 may have differing numbers of light sources (as well as both discrete and distributed sources), depending on their individual brightness, emission divergence angle, packing density, size, mounting geometry, and power-consumption. As an example, using medium brightness small 3 mm diameter LEDs, we would expect to have many light sources, for example on the order of 24 to 60. Using super-bright 3 mm or bigger (e.g., 5 mm) LEDs, one can make do with more like 12 to 30 light sources for the FIG. 1 depicted medium to large-sized assistive device. Of course, this also depends on the device size, with anticipated smaller devices needing fewer lights to maintain the same overall illumination intensity. The present inventors prefer the use of super-bright cylindrical light sources as they provide the most light in a small footprint and modest current. Arrays of surface mount LEDS are also attractive, even ones which are not super-bright, so long as they can be provided in numbers sufficient to overcome that.

The types of light source technologies useable in the present invention cover basically any available light source technology. These certainly include, for example, LEDs (light emitting diodes), filament-incandescents, solid state laser chips (including vertical cavity versions), halogen bulbs, arc-lamps and all manner of discharge lamps, fluorescent-based sources, phosphorescent-based sources and OLEDs (organic light emitting diodes). Also included are very short pulsed lamps such as flashlamps.

The light sources may emit constant light (e.g., DC or AC powered) or pulsed (e.g., DC or AC powered) light and those of the same or different color or mounting position may be operated simultaneously or in a temporally interdigitated, interleaved or overlapped manner. The user will perceive time-averaged or fusion-rate colors and intensities from mixed-color and pulsed lights in the known manner, presuming the pulsing frequency is above the beginning of the known visual fusion threshold in the 10-30 cycles/second regime. The invention may also be used with pulsing or switching lights wherein switching or pulsing frequencies are below the perceived visual fusion rate such as at 1 to 10 cycles/second. This is actually beneficial when the practitioner wants to discretely see the difference in contrast between two different lighting wavelengths and/or two differently situated light sources. The eye is very good at detecting and interpreting such variably-illuminated features, particularly in regard to their location and true dimensions.

Of particular utility is the time sequencing of light sources that deliver light to optically-contrasting targets of interest typically from different angles or depths (and/or from different colors from the same or different light source locations). This serves to help the user judge the feature position and depth in/under the observed tissue regions in slot 2 and radius 5. Such time sequencing is best done at visual fusion rates of 15 to 30 frames per second or faster if visual blending is desirable—and at a lower frequency (e.g., 1 to 10 cycles/second) if discrete states of differential lighting are to be compared or seen. Differential lighting effects can also include, as opposed to on/off operations, intensity-varying or color varying operation of two or more light sources relative to each other or of one or more light sources.

We note that surface-mount LEDs and light sources can be much smaller than cylindrical type LEDs or light sources and therefore one can fit more of them in a smaller and/or thinner and/or deformable package. They can also more easily be arrayed, perhaps even having different beam angles and in N by M rows/columns. They can also be mounted to a flex circuit or PC board and provided to the device during manufacturing as a pretested subassembly. A shaped flex circuit (not shown) with surface mount LED chips provides an inexpensive and compact manufacturing approach.

The present inventors make a further note regarding light sources and how to employ them. We have mentioned that viewing tissue features at two or more simultaneous or sequential wavelengths can be useful. Another way to do this is to populate a viewing aperture or window region 2 with different color lights at different window 2 locations. Now, when the device is physically scanned upon the tissues, one not only translates/rotates the skin/tissue under the window but also can alternately pass the two or more different colors over feature(s) of interest. So this is essentially color-switching via repositioning. Of course, our previously mentioned methods of electrical switching among bulbs or among the different colors of a given bulb can also be employed, in a fixed device position or also across scanned positions.

Moving now to FIG. 2, we see a three-dimensional perspective illustration of the example inventive device 1 of FIG. 1. Again the slot-defining arms 3 and 4 are easily seen having, in phantom, a number of internal light sources 7 injecting light into the tissue at an injection angle shown in this example to be about 45 degrees from skin-normal. Although not shown in FIG. 2, but in a manner similar to FIG. 1, the light sources are ideally also situated in the radius 5 region. We note that in FIG. 2, the light sources 7 along the straight arms 3 and 4 all have their central beam alignment axes 7b directed at a common tissue-subsurface focal line at depth D. The device 1 may be utilized to help guide a procedure or intervention such as a needle-stick or puncturing biopsy and that procedural implement is delivered most easily from an approach direction depicted as approach direction 14a in FIG. 2 (also seen in FIG. 1B). However, the device 1 may be utilized with any desired implement-approach direction(s) and/or implement-entry position(s), which does not necessarily have to be from an open-ended slot 2 (or, alternatively, a depression) as shown in FIGS. 1 and 2. For example, a needle-stick could be done from a midslot 2 position allowing for actual needle penetration in radius region 5. Further, the procedural implement might even be or include the practitioner's one or more fingers. Practitioners frequently place their finger(s) on patient skin or tissue such as to clamp or manipulate rolling veins, for example.

Most applications of the device 1 will involve a procedure being performed or delivered while the device 1 is on, juxtaposed to or held near the skin/tissue. However, the pre-sent inventors anticipate some applications wherein no immediate (or any) procedure is performed at all or wherein a procedure is performed after a feature of interest is located and the device has been removed or moved aside. By “procedure” here we mean all types of procedures including invasive ones, noninvasive ones and therapies such as electrotherapy and magnetotherapy or magnetotherapies.

In FIG. 2, we depict a mountable, attachable or flip-up filter and/or blood splash shield 10 in a folded-open (unused) state. We have found that filter/shield 10 can be very helpful in filtering out interfering room-light or other ambient wavelengths, if any. Note that the depicted shield 10 can be utilized while conducting a needle-sticking as from approach 14a as by closing or pivoting it somewhat so skin region 2 and any procedural implement or needle (not shown) can be observed through it as from the hinged side. In the FIG. 2 depiction, the filter/shield 10 is shown folded or swung back so that we can see the full second tissue portion in slot 2. Again, during use, filter and/or shield 10 would likely be folded somewhat over the slot so the slot 2 tissue features of interest and procedural implement(s) can be observed through it yet the guided implement(s) (and/or finger(s) if desired) can still be inserted into the slot region 2 along approach 14a. A filter and/or splash shield may also or instead be provided as face or eyewear and we consider this option to also be part of the device, particularly since the desired filter color is entirely dependant on the device light source wavelength(s) and/or any room or other ambient light wavelength(s). Typically, a filter will selectively block ambient light components from falling upon the skin such as fluorescent light components or sunlight components.

Further, we see the near sides of two straps 11 in FIG. 2 that can serve a number of useful purposes as follows:
1) to simply hold the device 1 upon the skin, limb, organ or tissue in a given fixed (at least relative to skin or tissue surface) location;
2) to hold the device on the skin, limb organ or tissue while moving it from location to location, thus allowing for some sliding, bending or rotational freedom of the device housing arms 3/4 (at least relative to the skin or tissue surface);
3) to distort the skin, limb, organ or tissue to improve an optical contrast of a feature, including providing such distortion independently of the device a itself. Thus, the straps 11 may pull on the device when tensioned, or they may completely enwrap the limb, organ or tissue and pull only on themselves, the device being attachable to their surface in any convenient manner;
4) to manipulate blood flow in a tissue, skin or organ subsurface or subsurface lumen; or
5) to support TENS electrodes, other electrical or magnetic therapy electrodes or drug-delivery entities to be described.

We explicitly note that desired device motions may be relative to the skin or tissue surface and/or relative to the skin/tissue-underlying tissue (e.g. deeper veins or tumors) and these may move somewhat independently (or not) depending on how tightly the device is attached or otherwise juxtaposed. Veins can move somewhat relative to their overlying skin/tissue or vice-versa. Thus, when we implement device/tissue motion, it may be motion of the device relative to adjacent skin or device/skin coupled motion relative to deeper underlying tissue or lumens. Such relative motion variations are apparent given the optical contrast such that the practitioner can manage them as by controlling slippage (if any is to be allowed) of the device to skin interface. This can be done as by controlling strap(s) tightness or device skin contacting-forces.

It will be appreciated that the strap(s) 11 may be permanently attached to the device 1 or temporarily attached. In both of these cases, they may be attached to device 1 before or after device 1 itself is juxtaposed to tissue. In an alternative approach, the straps may be pre-attached to the skin/limb/organ, the skin/limb/organ possibly favorably distorted, and then the device 1 is attached to the pre-attached strap(s) 11. In any case, the straps may themselves totally enwrap the limb/organ or they may partially enwrap the limb/organ in the cases wherein

a) the strap(s) are attached to the device sides as shown, or

b) the “straps” are actually pliable clamping or pinching arms (not shown) and do not need to be completely enwrapped and co-linked to perform a useful limb or tissue gripping and/or distortion function.

We note that depicted strap(s) 11 may be stretchable, unstretchable, tapelike, bandlike, chainlike, adhesive-like or suction-like. The device housing itself may also or instead employ such attachment or friction-controlling means such as adhesives or suction between it and a skin or tissue surface. They may also be semi-rigid or elastically bendable and be more like clamps or clasps (not shown) than flexible straps (shown). They may or may not pass all the way around the limb or organ of interest and interlock to each other as described. However, a common approach will be stretchable straps (shown) that bind or otherwise adhere to each other's ends to achieve an at least temporary device 1 mounting. The straps, particularly if they are more in the form of separate malleable or elastic arms (not shown), will possibly not interlock as, for example, on the limb far side such as for the second device example of our cuff device. The arms might even be an integral part of the device 1 housing, for example, and such arms might form an enwrapped enclosure by buckling or clasping to the device housing upon snapping-shut. We noted that the straps/clamps may be pre-applied to the skin/tissue before the device 1. In that case, when the device is applied to the pre-applied straps, it might be rigidly held or it might be loosely held such that device/skin or device/skin subsurface tissue scanning motion is still allowed. The present inventors specifically anticipate an embodiment wherein the device is loosely (and possibly also tightly) mountable to the limb, the loose condition being a scanning condition. By “loose” we mean movable with some effort without dismounting, relative to one or both of surface skin or tissue/lumens underlying skin. Depending on scanning and fixation requirements for the intended procedure, one may arrange for the straps to either be slippery (e.g., Teflon® fabric) or non-slippery upon the skin surface.

In FIG. 2, we depict in phantom lines an exemplary rechargeable lithium-ion or nickel metal-hydride battery(ies) or battery-cell(s) contained inside device 1. The battery also may have an adjacent power or recharger jack 13 that can allow for an external device power source or recharger. Of course, the battery may instead be a fuel cell, an energy-storage super capacitor or even non-rechargeable batteries. We have also recently seen on the market a variety of portable “Power Packs” such as the UPB10 “Mobile Power Pack” from APC of West Kingston (RI). The attraction of this device is that it can power a power-consuming product via its PC industry-standard USB port. Thus, if our assistive device were to have a USB port, it could be powered by this APC device and/or from any PC with a USB port. A mobile power-pack might also have a non-USB connector to our inventive assistive/guidance device of course.

We depict in FIG. 2 a subsurface lumen 12 within the slot 2 as it would be visible with the device-produced improved optical contrast. Generally speaking, the device 1 will be scanned relative to one or both of skin or tissues beneath skin (while strapped or unstrapped) to find a desired feature such as a lumen 12. We note that such scanning may take place any one or more of:
a) Scanning the device without any straps (or a device designed to be strapless), which is the least preferred.
b) Scanning the device with straps pre-attached to it but not yet (or not ever) tightly-un-movably clasped to tissue or limb.
c) Scanning the device with straps which are pre-attached to it and pre-attached or clasped (perhaps loosely) to the tissue or limb (modest scanning forces if any).
d) Scanning the device/strap combination wherein it is snugly (but not un-movably) pre-attached or clasped by the straps to the tissue (may require significant scanning forces depending on snugness).
e) First mounting the straps to distort the tissue to either enhance an optical contrast or manipulate a bloodflow then mounting or juxtaposing the device 1 to or over the pre-attached straps, the device preferably being affixed to the taut straps in a movable manner, at least initially with a mating mounting mechanism. Note that device fixation to limb-pre-applied straps might be loose in that some scanning is permissible with (at most) modest force, or snug such that scanning is not allowed or encouraged (but might be possible with the larger forces). By “loose” we mean the device is movable with respect to the skin and/or underlying tissue regardless of whether the strap(s) moves with the moving device, i.e., the device could be attached to the strap(s) on a slider that permits mounted device sliding and possibly slider-locking. Note that in that case, the device moves and/or locks its position/orientation relative to the strap(s), perhaps even after the straps have been snugly tightened.
(f) Scanning the device at a skin standoff gap or distance from at least part of the device, the gap being controlled in a freehand manner or utilizing a spacer means such as a compressible gasket.

Again, by “strap” we include all manner of locking clamps or members 11, which may have their own malleability, elastic or spring nature, limb-grasping or locking means, clips, buckles, snaps, self-adhering portions, or zippers. These therefore do not necessarily have to clasp or fasten to each other in order to achieve limb or organ fixation; in fact, there may be only one which wraps mostly around a limb or organ on a housing-hinge, for example.

The strap(s), especially in the case of their being adhesive or microscopically (e.g., Velcro®) interlocking limb or tissue adhering members, might be disposable. They (or it) may also be situated partially, mostly or entirely under the device in the device/tissue interface and might therefore include holes or transparent regions for the light sources or for the viewable contrast region(s). Such strap(s), adhesive or otherwise, may also support a TENS or other electrode or magnetic therapy means or drug means such as for pain-reduction or lumen-dilation described further below. We also include in the scope of the invention the device delivery of warmth or cooling to tissue of interest used for purposes such as pain-relief, accelerating or activating drug activity or uptake or for encouraging lumen and/or capillary bed dilation or constriction. Such dilating warmth may come, for example, from our assistive light sources, from separate light sources (e.g., infrared), from the waste heat of the device itself, from dedicated heaters or coolers in/on the device or from a pre-warmed or pre-cooled device as pre-warmed or pre-cooled in a pre-warming or pre-cooling device-tank, holder or thermal medium.

The present inventors anticipate variations upon the device wherein one or more arms or members 3 or 4 can be flexed, bent, or twisted into a desired formable or positionable shape or geometry such as to more suitably conform to or favorably align with or be juxtapositioned to a tissue or limb of interest. Such deformation may be in one-, two- or three-dimensions and might be elastic and/or plastic in nature. Such deformation(s) may itself provide limb or tissue fixation capability, thus possibly eliminating the need for separate (from arms 3, 4) fixation straps 11. Note that this would comprise a self-fixating device 1. An example of this is that wherein arms 3 and 4 are elastically wrappably deformable around a limb or organ portion or skin volume such that they self-clamp to the limb, organ or skin. Our mentioned cuff device could easily be made self-clamping by making it in the form of a mostly closed or totally closed or closable bracelet or armband. By “conform” we mean any one or more of: (i) having minimal lateral surface light-leakage, (ii) having a set of light sources creating useful contrast from a desired set of injecting positions/angles, (iii) providing a loose or snug device fixation action by conformably squeezing a tissue or limb, (iv) causing a desired state of skin/tissue distortion for optical-contrast and/or flowable lumen manipulation reasons, or (v) applying TENS or other electrotherapy electrodes, magnetic therapy fields, a drug-source, a vibratory source or a heat-source to tissue utilizing a device conforming behavior or force.

We note example bending arcs 3a and 4a depicting two arms 3 and 4 which might be bent (or simply rotated), at least temporarily, to allow for better device conformance and/or a more desirable slot shape 2/radius 5 second tissue portion observable-contrast region. Note that such bending, distortion or rotation may be in one or more planes (one plane shown) and may involve malleable, plastic, elastic, hinged, sliding or pivotable motions of housing parts or of light sources within. It could also involve substitution of parts as for a kit containing a variety of arm geometries.

The first tissue region is that where the light sources are doing their light injection under the arms 3 and 4 and adjacent the radius 5 slot region. In the depicted device of FIGS. 1 and 2, this first tissue portion(s) is under the arms 3, 4 and radius 5 edges at the tissue interface where the light from sources 7 is injected into the tissue at the device/skin contacting or juxtaposed interface.

Practitioners doing needle-sticks will frequently utilize tourniquets or other blood flow stoppage means to make subsurface lumens more visible and/or more stickable. Within the inventive scope is the use of our fixation strap(s) or attachment means 11 for that tourniquet purpose and/or for the general purpose of distorting the tissue to favorably affect a desired aspect of procedurally-useful optical contrast or stickability. Recall that such fixation may, in part or in total, be offered instead by deformable or pivotable members themselves such as arms 3 and 4 which could capture tissue or a limb between them.

We note in FIG. 2 a tissue or skin-element 15 at or near the tissue surface and resident in the optically contrasted second skin portion slot-like observation region 2 such as a tissue 9a element 15. Shown is a tension T applied to that (and other) skin or tissue elements by the mounting of the device 1 and/or its straps 11. We stress that the tissue in the slot 2 region (surface and subsurface) can be favorably manipulated in a number of compressive, tensile or shear directions by manipulating the strap(s) 11 tightness and/or by moving or deforming device 1 member arms such as by deforming or pivoting arms 3 and/or 4. The skin/tissue can even be bulged or extruded out of (up into) the slot toward the practitioner thereby allowing for more light to be delivered underneath the features of interest, particularly shallow features of interest. This allows for tangentially directed (or even somewhat upward directed) light to be delivered into subsurface skin or tissue-light that would otherwise possibly be a surface-leakage problem for non-extruded skin. This scheme can cause some lateral shadowing though and we therefore also offered the operational mode wherein the device is gapped (with peripheral spacers for example) from the skin to avoid unacceptable amounts of extrusion. In any contacting device, some tissue deformation will take place and moderate amounts are usually acceptable.

Recall that we may promote beneficial lumen or vein dilation using the TENS, NMES, other electrotherapy or magnetic therapies and/or drugs as well or instead of using physical tissue distortion means such as the strap/device loading.

The device 1 is comprised mainly of a housing which has one or more appendages, arms, or slot/window (region 2) defining perimeters possibly deformable or movable, which can define an observable second skin portion(s) such as a region 2. Observable second tissue region 2 does not necessarily have to include a lateral opening (shown in FIGS. 1 and 2) or depression (not shown) as long as appropriate device heights and tapers allow for any needed procedural implement to approach and perform its function(s). Note that the shown open end of the slot defined by arms 3 and 4 could alternatively be bridged by a low-profile thin skin-adjacent member (not shown) that allows the arm(s) to be locked to it in a deformed or nonparallel position or in a position wherein the device 1 is clamping tissue, for example. The device housing will typically be at least partly polymeric in nature and be formed using molding, casting or extrusion techniques. The housing will frequently have an internal metal or other semirigid skeleton or mounting features for the light sources, their electrical connections and any heat sinking features and a battery(ies). Typically, at least some circuitry will be provided such as LED driver chip(s) or TENS circuitry. Other circuitry may control or monitor battery charging or charge-state, light source intensity, light source on/off or pulsing, coordinated light sequencing or the operation of an integrated electrical or magnetic therapy electrode or implement as mentioned earlier. In a preferred approach, we utilize super-bright LEDs in surface-mount form (not shown) or in cylindrical bulb form (shown). Alternatively, one could have a distributed light source (not shown) that is routed around the tissue contact surface but which itself has one or a few possibly skin-remote illuminators feeding it. An intrinsic advantage of surface-mount light sources such as surface-mount LEDs is their low profile and small size. This allows for a thin device 1 and also allows for numerous such chips to be oriented at different beaming angles (if desired) to tissue. A low profile (thin) device 1 makes observational and procedural work with it even easier due to the wider angular field of view of the practitioner. It is also more easily pocketable and has easier implement access.

We include in our inventive scope the inclusion of any blocking or masking feature(s) which favorably psychologically prevent the patient (or practitioner) from directly viewing the procedural site for at least some period of time-if that is desired.

We also include the option wherein a disposable entity is a light-distributor, such as a disposable arm that distributes light and when mated to the housing is optically coupled to a light source(s). Only the light-distributing element might rest on or juxtaposed to tissue. The powering housing might be situated off of the limb.

Movable or deformable housing members or arms such as 3 and 4 may be elastically or plastically deformable or may even be hinged or mounted on sliders. They may be lockable in various states of distortion, position or shape by a dedicated locking means (not shown) or as by a condition of strap or hand/finger-loading of the device 1. Any internal electrical wiring that connects to the light sources may be in the form of any one or more of flexible circuitry, printed circuit boards or discrete wires or cabling (none shown for simplicity) for example. Fiber optic or light-transmitting conduits, reflectors, gratings, other optical elements or arrays thereof or diffusers may also be routed in the housing as for delivery or direction of light to or from one or more discrete or distributed apertures or light-emitting windows.

Generally speaking the device will be at least wet-wipeable as with a damp disinfectant or sterilizing wipe or towel. Other hardier models may be designed to be further immersable or perhaps even autoclavable or treatable by hydrogen-peroxide sterilizing procedures. Further, looking at the bottom (skin-contacting when used in device-contact mode) first tissue-portion surfaces of the device where the light sources emit their light, it will be desirable to have a smooth surface with the possible exception of any depicted light-dam or light-gasket 6. Such a smooth surface can be provided as by a smooth optically transparent window layer (not shown) overlying at least the light sources. In some designs, this window layer may serve the above-mentioned skeleton function wherein light sources mount into that skeleton or backbone layer first during manufacture. It is advantageous from the cleanliness and comfort points of view to have the bottom device surface be smooth or at least have no reentrant cracks or slits between mating parts.

We now temporarily go back to FIG. 1 to note device radius 5 therein. It will be appreciated that in order to use the device in the crook of the arm or in other limb joints it is advantageous to have a device/tissue mating interface that prevents light leakage even for the non-flat surfaces of such joint regions. Within the inventive scope is a radius such as 5 being adjustable in any manner including by substitution of parts, hinged or pivotable parts, or malleability. The light gasket 6 may be rigid or might be soft and deformable as for an inflatable light gasket 6 or deformable foam gasket 6. It might also comprise a separate (from the device) disposable patch or article. Note that we have now taught tissue/limb fitting conformance by one or both of two different mechanisms, i.e., a) a preshaped device and b) a shapeable device. These two mechanisms may operate in the same or separate device regions. Of course, the tissue/limb itself also conforms to some degree if physically loaded.

Preferably, it is anticipated that with a few hours of overnight recharging, the device can be utilized for most of the clinician's workday without a battery change or help from an external power source/recharger. However, the device may also be operated as by changing out weak batteries or by plugging in a power source and/or recharger into a connector such as 13 (with or without the battery present). A battery-capable or self-powered device capability is preferred. Obviously, the total useful light emission time will scale with the total integrated light intensity and battery capacity.

We anticipate the possibility of any one or more of the following features being utilized in/on/with the device apparatus or as part of the device application and use method:
a) A skin/tissue presence/absence sensing switch or sensor which turns the device on or otherwise allows power consumption only when the device is skin mounted—this could, for example, be a capacitor, thermistor, thermocouple or LED/photodetector.
b) An intensity mode selector control which allows a practitioner to select among two or more optical-intensity or luminance operational states which result in different rates of power consumption.
c) A color or color-mix selector or control that allows the practitioner to select among one or more separate, sequential, combined or interleaved colors.
d) A light source (and/or audio) operational mode designed to amuse or capture a child's attention. This could utilize the same light sources and involve looking at the skin-face of the device (at a safe intensity of course) or at the top of the device while it is skin coupled, some light purposely being leaked or routed to the top of the device at least during this amusement-supporting operational mode. Such playful light might even be projected on a wall or desk. The device might even project an image of a cartoon character on the child's arm.
e) A simple room or ambient light(s) on/off switch or intensity (dimmer) adjustment switch.
f) Manipulative handles, grips, locking means or guides such as accompanying movable members such as 3 and 4. This also includes any features provided on the device/straps that serve(s) to control or guide a practitioner's finger(s) or hand position(s). As an example, the device could include finger-slots or depressions whereat the practitioner's fingers lie during device manipulation or during procedural delivery.
g) Adjustable and/or replaceable optical filters and/or splash shields 10.
h) A preferably attachable/detachable camera, imaging chip, imaging lens or imaging fiber-bundle particularly useable for infrared imaging and redisplay as visible optical contrast, but also or instead useable to provide visible imagery. Visible redisplay of invisible infrared imagery could be done as by using a digital light mirror projection chip (Texas Instruments DLM) or a scanned laser projector, for example.
i) TENS, NMES or other electrotherapy electrodes or magnetic means for pain relief and/or lumen dilation, particularly disposable ones that get their electrical power/signals from the device 1 battery and/or electronics therein.
j) Pain relief drug delivery as by a pre-saturated patch that is placed in/under the device or as by a separate drug-laden swab or spray. These are device specific in the sense that they are relieving a specific pain type and pain location related to the use of the inventive device.
k) Pain relief, pain distraction or needle insertion easement (reduction in required force) as provided by vibration as delivered into the skin from a device mounted or device-supportive vibrator. Such a vibrator would likely be similar in nature to a cell phone vibrator.
l) Pain relief or lumen dilation as provided by device-supported or device caused heating of skin/tissue.
m) Other therapy electrodes or magnets/electromagnets delivering other electrically-based and/or magnetically-based treatment or therapy.

Returning to FIG. 2 now, we note that, although not shown, one may integrate into the device 1a vibrator element whose purpose is any one or more of: a) distract the patient or relieve his/her pain when about to be or being needle-stuck as mentioned above, b) ease the mechanical effort of an actual sticking or puncturing event, or c) help distribute a delivered drug into tissue. We note that such vibrators, like those used in cell phones to alert the owner to a call or message, consume little power and take very little space.

Let us now discuss the observable contrast region slot 2/radius 5 again. The pre-sent inventors anticipate in many embodiments this observable second tissue portion being entirely open and unencumbered (as shown in FIGS. 1 and 2) such that a needle-stick or other skin/tissue procedure can be carried out anywhere within it without having to move the device 1 (assuming the feature of interest is already located somewhere within the observable contrast region). On the other hand, the practitioner might want to, once a feature is located, reposition the feature of interest to a particular sub-region of observable region 2/5. This might be for reasons of the easiest stick-access and/or because the contrast lighting behavior is somewhat different in different sub-regions of region 2. Recall that during such repositioning, the device may or may not already be attached, snugly or loosely (movably) to fixation means such as 11. In any event it is the device 1 that is being positioned relative to the observable feature(s) and that such positioning will involve at least some relative motion of the device 1 and the features(s). To do this might involve absolute motion of any one or more of the device, the skin, a procedural implement (if any), the practitioner's hand(s) or finger(s), or the straps or the underlying tissue/lumens.

Some embodiments are anticipated wherein observable region 2/5 has some bridging material elements or members (not shown in Figures). The term “bridging” means spanning across some or all of a dimension of the observable region 2 either in contact with the tissue or above the tissue. Presuming these bridging elements are optically transparent, then they will not interfere with the act of finding or locating the skin or subsurface tissue features of interest in the second observable contrast region(s). Such bridging features could, for example, be preferably transparent members which link or lock arm 3 to arm 4 at chosen adjusted or lockable slot widths or shapes. They could act as optical filters as well along the lines of 10. Unless these slot-bridging members are themselves stickable, which they might be, then one would move device 1 or a portion thereof (relative to tissue or lumens) to allow such sticking of tissue which was previously under such a bridging feature. Such bridging features may also comprise or include light source regions such as distributed light sources or transparent diffusers that may even be stickable through the bridging material (which may also be disposable). Note that in that case at least some first tissue portion and second tissue portion are the same portion. In one preferred case, a transparent bridging member is a distributed light source having its powering injecting light source off to the side such that the entire bridging feature, member or plate is transparent or translucent and light-injecting. We include in the inventive scope a bridging feature being any one or more of: (i) part of a strap 11 or other device retainment or fixation member, (ii) being disposable, (iii) being stickable as for a thin or perforated polymer or fabric, (iv) being a source of light, such as it being a light distributor (including even being the only light distributor), (v) being adhesive or suctioned in nature to the skin and/or to the device, (vi) being an optically transparent sheet of polymeric or fabric material as utilized, the sheet or fabric being either rigid or conforming, (vii) supporting or controlling a motion or distortion of an arm such as 3 or 4 or providing locking features therefore, (viii) serving as any part of a strap 11 system or anchor therefore, (ix) serving as a TENS or NMES electrode, other electrically-therapeutic or treatment electrode, source of magnetic therapy, source of drug, source of heating or source of vibration, (x) serving as a means to guide an implement, steady an implement, hold an implement or mate with and/or stick-protect a practitioner's finger(s) for any purpose, xi) being in contact with tissue or being raised off the surface of the tissue, or xii) being a tissue marking or measuring means.

It is expected that the inventive device 1 may be utilized anywhere on the body such as on an arm, leg, hand, wrist, ankle, neck or breast. It may also be utilized on exposed organs such as during surgery to find subsurface features or inserted invasive implements of interest or importance. Thus it might be inserted in a wound or body cavity, natural or otherwise. It could also be integrated onto the business end of a scope such as a colonoscope, gastroscope, bronchoscope or laparoscope and utilized remotely or by a robot.

The present inventors have also noted that the optical contrast provided includes optical contrast of the procedural implement itself, such as a view of the needle within the tissue and/or above the tissue. We include in our inventive scope the assistive/guidance device being used to visualize a needle or procedural implement below the skin/tissue surface. The present inventors have noted that needles can be seen to some depth as by a combination of toplighted/sidelighted reflection and bottomside light blockage or backlighting. One might even utilize a syringe lumen or other inserted implement as a light pipe in order to provide needle-tip tracking light on the needle itself. An invasive implement or needle might be made maximally reflective at the devices illumination wavelength(s) to enhance its subsurface visibility.

The present inventors emphasize that inventive assistive/guiding device 1 may be used for invasive or noninvasive procedural support. The “procedure” may involve an optical device exam alone, an optical device exam in support of a noninvasive or invasive treatment, or even delivery of a therapy alone without necessarily utilizing the optical exam capability. Many users will combine an optical exam with a treatment and many of those will have the treatment be optically-guided or assisted.

The practitioner, upon identifying a feature of interest using the device, may need to puncture or otherwise compromise a tissue/skin integrity or may not have to, either immediately or at a later time or date. An example of not having to disturb the tissue (at least not at the time of device observation) might be the device-assisted inspection of varicose veins to judge their state of disease. The practitioner might then recommend a non-surgical or surgical intervention or drug intervention be carried out on a different day(s) with or without the help of the device.

The present inventors also anticipate other valuable device embodiments such as:
1) Having the filter and/or splash shield 10 consist of one or both of a) a head-mountable or eye wearable component, or b) a disposable polymeric sleeve wrappable around or covering some or all of the device, the limb or any device portion.
2) Having a disposable filter, splash shield or wrapping bag or combination part of two or more of those.
3) Having a room-light or ambient-light dimmer or on/off switch accompanying (or mounted on/in) the device with which to dim or turn off/on the room lights. This may be a wireless entity. It would not necessarily eliminate the need for a filter but might do so.
4) Having color (wavelength(s)) selectable filter choice(s) or filter opacity choices.
5) Having detachable arm(s) 3 or 4 that are of different sizes, shapes or compliances or that have different light sources. a variety of arms possibly with different fits, compliance, size or wavelength may be provided in a kit. In this option the arms are probably electrically and/or optically connectorized or coupled to the parent housing portion.
6) Having multiple device designs 1, device components and/or straps 11 in a kit to address a multiplicity or range of patients or a range of anatomical parts. These may be of different size or shape or of different light source design.
7) Utilizing human non-visible light sources such as infrared or ultraviolet sources likely combined with an imaging means which presents the imaged (or excited) contrast to the eye on a screen or on a head mounted or eye mounted display in a converted human-visible manner. This approach may easily utilize an imaging chip that is capable of imaging at one or both of human-visible and human-non-visible wavelengths, the chip ideally being mounted on/in the housing as mentioned above. Numerous CMOS and CCD chips have both infrared and visible sensitivities as do compound semiconductor imaging chips. An advantage of near-infrared or NIR is excellent skin penetration and lumen contrast at the cost of requiring a NIR imager (unless the ingoing NIR excites a visible optical excitation in the tissue or in a contrast agent). (Note that the device might also employ warming IR, which is not used to do imaging.) Non-visible illumination may also employ contrast agents that emit in the visible or non-visible.
8) Having replaceable or movable light sources or angle/divergence-adjustable light sources.
9) Having floor-drop protection features such as rubberized device corners or a rubberized or springy housing, arms or skeleton.
10) Having device feature(s) that mate with or lock with a limb or tissue clamping strap or element or with a TENS, other electrotherapy electrodes, magnetic therapy means or cables therefore.
11) Having the device 1 deliver a chemical medium such as a skin-coating or skin-permeating gel or liquid that improves a skin or tissue optical transparency or a drug that deadens a real, anticipated or perceived tissue sensation or patient's tension or that treats a disease or disorder.
12) Being utilized with a separate or included skin stripping means utilized to strip off at least some skin surface dead cell layer to improve a desired optical contrast. This may be an adhesive or abrasive patch preferably provided as a disposable. It would not necessarily have to be used during optical contrast creation and might be used only before device fixation or use.
13) Having an optical polarizer or antireflection coating in an optical path to reduce glare or to improve an optical contrast.
14) Being utilized to deliver an excitation light, probably largely human invisible, to, for example, a skin or subsurface optically excitable or fluorescent contrast agent, the optically excited agent light-output assisting or creating the desired optical contrast in one or more manners, the optically excited agent output-light preferably being human visible or even more likely visible to humans using an excited-light sensitive camera means and a connected human-visible display means. An optically excitable agent may be systemically or locally delivered in any manner including being provided from the device itself, from a device-guided implement, or from an adhesive-patch or drug-patch therefore. Such excited light is frequently, for example, in an infrared, ultraviolet or visible wavelength range. That light would probably require aided observation using an imaging and redisplay means. Excitation mechanisms might include fluorescent mechanisms, phosphorescent mechanisms or quantum-dot (excitable nanoparticle) type mechanisms.
15) Being utilized to also deliver preferably non-invasive electrical stimulation or pain suppression to tissue regions of interest as for example using the known “Transcutaneous Electrical Nerve Stimulator” technology or “TENS” or NMES electrotherapy. The delivery may be associated with a guided/assisted procedure or a pre-existing condition, for example. This may be done as by one or more electrodes integrated into the face of the device 1, adjacent the device or on other appropriate skin, tissue or limb sites that will dull pain in a region where the guided sticking procedure is implemented. Such electrode locations may even be opposite the inventive device on the limb. Such TENS or NMES therapy may be delivered separate from or in combination with a needle-stick or guided procedure. In fact, we also include the possibility of a procedure-related needle or penetrator acting as a TENS, NMES (or other electrode-based treatment) electrode. The optical contrast provided by the inventive assistive/guidance device might instead be utilized to determine where to deliver TENS, NMES or other electrode-based treatment, i.e., the TENS or NMES treatment being the device guided procedure-whether of not the TENS or NMES electrodes are device-integrated or not. In most cases, however, TENS or NMES would be utilized to reduce or avoid perceived procedure-related pain and anxiety.

The rationale for using TENS or NMES technology for pain is based on the following: It is well known that intensely massaging a pain site tends to reduce the perception of pain. This is because in the spinal cord there is a “pain gate” that serves to filter nerve impulses, only permitting the majority of impulses from a certain class of fiber to travel through the spinal cord and to the brain. For example, if pressure impulses from A delta fibers outnumber painful impulses from C pain fibers, then only the pressure impulses will be received by the brain. In this way, by applying strong pressure to a very localized site, pain signals will not successfully be transmitted through the pain gate and hence the patient will perceive little or no pain.

TENS, NMES or any other electrode-based treatment electrode(s) may be applied to the skin as by mounting them under or adjacent the device and/or its fixation straps 11. They may be powered by their own supply or by electronics and/or power provided by the device 1 as through an electrode jack similar to the jack 13. The present inventors have found that continuous TENS electrical excitation seems to give somewhat more patient pain-relief than pulsed excitation. We have also found that it is important not to press too hard on the operating TENS electrodes as this can actually cause pain. Thus, we anticipate lightly loaded TENS-pain electrodes or self-adhering electrodes being preferred. In terms of TENS electrode(s) placement, what is important is that the site of the actual procedure implementation benefit from the associated electrical TENS current but that will frequently be possible having the electrodes located away from the actual procedural site—such as on the device sides or further around or along the limb.

We emphasize again that device-integrated therapy or treatment delivering electrodes may be utilized for purposes other that pain reduction, whether the pain is needle-stick associated or is from a preexisting condition such a varicose veins, a pulled muscle or a pinched nerve. Thus the device is a generic electrotherapy, electrotreatment and magnetic-therapy/treatment tool as well. A second such purpose is the mentioned lumen dilation. Inventors have demonstrated both TENS and NMES lumen dilation.

The overall size of the device may be, depending on patient size and bodily application to man or animal, from an inch or so up to ten inches or so in its maximum length, width or diametral dimension. Most useful devices will be in the 1.5 inch to 8 inch maximum dimension range. A device skin-abutting or juxtaposable surface(s) may be flat or curved, rigid or conforming. The present inventors have fabricated more than 15 good working devices generally like that in FIGS. 1, 2 and of the cuff design for limb use in these ranges and have found that devices with an overall maximum lateral length dimension of 2-6 inches are most desirable and are easily pocketable in a lab coat or even in a shirt pocket. The cuff design can even be carried by the practitioner on his/her own wrist if not in his/her pocket.

The second skin portion optical-contrast viewing region(s) may or may not have an access opening (or depression) on its end or edge (as shown in our FIGS. 1 and 2). One good alternative to an opening, as mentioned earlier, is a partial opening wherein a thin skin-adjacent bridging member bridges the viewing window or hole but is so thin that it does not interfere with any approaching guided implement (not shown). That bridging member may also possibly offer a locking or guiding action for movable or otherwise distortable arms yet may possibly be optically clear or translucent and narrow (and even stickable). It may also rigidify the device so it does not undesirably deform in some manner during limb or skin fixation as by the influence of taut straps 11.

Any portion of a second skin portion viewing hole or window 2 may be temporarily or permanently bridged by assistive bridging components (none shown in FIGS. 1 and 2). These may or may not be partially or entirely optically transparent or translucent and may be permanently attached, temporarily attached, hinged or movably situated to serve their function. By “bridging” in this context we mean attached to or mounted to at least one edge of the viewing window/hole at least temporarily and/or at least temporarily arranged to span any whole or partial portion of the hole or window 2. By a stickable bridging member we mean the member is stickable as by physical puncturing and/or by delivery of the sticking implement through a preexisting bridging member aperture, hole or slot. Such a stickable bridging member may be disposable and/or cleanable and is preferably separable from the device housing.

Examples of above-mentioned and further window/hole bridging components include:
a) previously mentioned distributed light means such as a (preferably) practitioner-transparent/translucent optical diffuser that primarily diffuses ingoing light downwards into tissues but may still allow for contrast observation therethrough;
b) as in (a) but wherein a device-guided procedure or needle-stick can be performed through the bridging material, as by puncturing the material with a syringe needle or by passing the needle through a preexisting hole or orifice in the bridge material designed for that purpose;
c) bridging members that serve to favorably deform or clamp tissue, to increase optical contrast, control blood perfusion, immobilize rolling veins, minimize light leakage, minimize bleeding, achieve a skin standoff height of the light sources or to provide device fixation;
d) as in (c) but wherein a device-guided procedure or needle-stick can be performed through the bridging member;
e) support one or more TENS, NMES or other electrotherapy electrode(s) or pain drug-delivery member(s) or pads, whether needle stickable or not;
f) support one or more other therapeutic or treatment magnetic-field application means whether or not stickable and whether or not any sticking is to be done;
g) support for an ultrasound transducer used to validate vein, artery or implement locations. Ideally this would utilize miniature transducer arrays such as CMUT “capacitive micromechanical ultrasound transducer” arrays of very low cost and might itself even be disposable;
h) provide any one or more of an optical filter, antireflection layer, lens, reflector, polarizer, imaging chip, splash-guard, practitioner needle-stick protector or component thereof;
i) provide a flexible light emitting or light injecting film, fabric or mat such as a thin polymeric OLED light emitter or a transparent light-diffuser or reflector;
j) provide a marker or measuring means used to mark or measure a site or feature of interest, the placed mark possibly being utilized after the device and/or mark-making means is removed from the limb or tissue, the mark being at least temporary;
k) provide a means to clamp or lock a deformable or actuatable motion of the housing or one of its movable arms;
l) provide a physical guidance, gripping or locking feature for a guided procedural implement and/or a practitioner(s) finger(s). This could even include fixation, mounting or integration of the procedural implement into/onto the device; or
m) provide a heating or vibrating means as for pain reduction, needle insertion easement or another therapeutic or treatment purpose.

Given that many of these capabilities may optionally be used with or on the device, the present inventors expect that the device may be provided in kits containing one or more of these additional optional components, some of which may be disposable. Other of these features, disposable or not, might be purchasable as optional separate items. We also expect that the practitioner may want to purchase such consumable components in quantity-preferably in individually sterilized packages in sterile bundles thereof. Web-based purchasing would be convenient.

We expect that the device will frequently be arranged, as taught, to support pain-reduction as for reducing the actual and/or perceived pain and/or anxiety of a needle-stick or other procedure-related skin disruption. The above described TENS and NMES electrode(s) on the face of the device or elsewhere on the skin surfaces can reduce such pain. Whether located on the device face, adjacent the device, or elsewhere on the skin one would likely utilize two or more cooperating electrodes that treat tissues, at least the procedure-related tissue between them or electrically coupled to them. Magnetic exposure may also decrease perceived pain.

Note again that pain treatment using the device might also involve preexisting pain having no association with pain caused or anticipated by a device-assisted procedure.

We emphasize also that device related pain treatment, such as by electrotherapy of drug delivery, may be for procedure related pain or preexisting pain issues such as pain from an injured joint or skin-burn.

A pain-relieving gel or cream as supplied in the form of an associated drug or medicament-laden wipe or patch may instead or additionally serve such a purpose. Such a wipe or patch may be used on the target tissue one or more of: (i) before device placement or juxtaposition to tissue or skin, (ii) after device placement or fixation such as after a feature of interest has been found using the device, or (iii) after or during delivery or performance of the device-assisted procedure (e.g., needle-stick, biopsy-stick) before or after device removal. Similarly, the device itself could be provided with a disposable pad that serves one or more purposes such as adhesive-mounting of the device and/or delivery of a pain-reduction or optical tissue-transmissivity improvement cream or gel. The device itself may include a reservoir or even just a wettable surface for such a drug or medicament. Both TENS and/or NMES electrodes and the drug may one or both also encourage lumen and capillary bed dilation.

An optical-contrast agent, if employed, would most likely utilize an optically excitable dye or nanoparticles that may be systemically or locally delivered, and furthermore may even be biologically targeted to specific molecules or diseased cells. Such excited optical contrast might also be excited from the natural tissue and body fluids themselves without an agent as some bodily constituents are fluorescent. In this manner, the assistive device may view skin and subsurface disease states that may then optionally be treated independent of the device, the device assistance being the finding of such disease state if it is pre-sent.

The device may be provided TENS and/or NMES-capable (or capable of other electrotherapy modalities), meaning it already has built-in at least some of the necessary connector(s), signal generation circuitry and power-source to drive TENS, NMES (or other electrotherapy) electrode(s). Thus using TENS, NMES (or other electrotherapy) would simply be a matter of plugging the electrode connector(s) into the device and switching the TENS, NMES (or other electrotherapy) capability on. We include in the scope of our invention the delivery of TENS, NMES (or other electrotherapy) treatment before, during and/or after a guided procedure is carried out. The device vendor may also opt to include the TENS or NMES support only on units specifically purchased to also support TENS, NMES (or other electrotherapy). This may be as by its omission or deactivation as well. The present inventors note that once electrodes are provided, a variety of electrotherapies (see terminology section above) can be provided by simply driving the electrodes differently using different software or firmware.

Pain reducing drugs that may be utilized in support of the device or its application method include topical rubs, balms, oils, lotions, liquids, creams, ointments, patches, gels and sprays. Ones that inventors have worked with include:
Rubefacients: traditional formulations based on salicylate and nicotinate esters, capsaicin and capsicum extracts and derivatives;
NSAIDs: diclofenac, felbinac, ibuprofen, ketoprofen, piroxicam, naproxen;
Local anesthetics such as lidocaine, benzocaine, and other “caine” anesthetics;
Capsaicin and capsaicin-derived products: such as Menthacin and Zostrix, mentholated products, salicylates (aspirin and NSAIDs), hydrocortisone and other corticosteroids, EMLA cream, and ethyl chloride spray;
Clonidine and clonidine also in combination with a second nitrate-containing species; and
Benzydamine, mucopolysaccharide polysulphate, salicylamide, and cooling sprays.

Any one or more of these may be used in combination, topically, subcutaneously or systemically, as the practitioner determines is safe. Further, any one or more may be used in combination with TENS, NMES or other electrotherapy or pain-relief, vibratory pain-relief, heating pain-relief or any other kind of heat-therapy or electrotherapy as also judged safe by a practitioner.

A tissue optical transmissivity improvement means, if employed, may include a chemical that permeates the skin or tissue such as glycerol, glucose, trazograph, propylene glycol, sodium lactate, butylene glycol, vegetal compounds or liposomes. Such materials are historically known to improve optical skin transmissivity. Another related known optical transmissivity improvement means is skin surface-layer stripping to get rid of the opaque dead skin cells on the skin surface. Either or both of these could be provided for use with the device including as part of the device itself as for a device adhesive pad which also delivers a transmissivity-improver. Likewise, the device itself, or a protective cover therefore, may deliver to the skin such a transmissivity improvement medium. Skin abrasion may be implemented using adhesive tape or an abrasive implement that may or may not be device-integrated. Included in the scope of the present invention is a pain-reducing drug or and device-delivered medicament that is also a transmissivity improver as is a pain-reducing drug or electrode treatment which relieves skin pain due to skin surface stripping.

The inventive device may even drive the transmissivity improver into the tissue as by an onboard heater, vibrator or ultrasound source or by electrophoretic or iontophoretic mechanisms. Any vibrator means may also be used to drive abrasive dead skin-cell removal.

What is claimed is:

1. A medical-procedure assistance or guidance device capable of providing substantially non-invasively-produced or excited optical-contrast of one or more of cutaneous, subcutaneous, subsurface or internal tissues, body-fluids, lumens or features that may require locating or invasive intervention, including such as for needle-sticking or needle-biopsy sampling, the produced optical contrast improving a practitioner's ability to one or more of locate, target or avoid said tissues, body-fluids, lumens or other features in support of an immediate or later procedure, treatment, therapy, diagnostic test or intervention requiring knowledge of said features(s) location(s) or properties, the assisted or guided procedure, treatment, therapy, diagnostic test or intervention which may or may not involve physical skin or surface-tissue penetration to or in avoidance of the located subcutaneous or tissue-subsurface feature(s), the device comprising:
an illumination or optical-excitation housing physically coupleable, adjacently-presentable or closely or contact-wise juxtaposable to a skin, tissue, limb or organ surface, the housing containing, including or supporting at least one or more discrete light sources and/or one or more distributed light sources, at least some of the light output of the at least one powered discrete and/or distributed source directable at least into one or more skin, tissue, limb or organ first-portion(s);
and
an optical contrast-improvement means,
the device providing improved optical contrast of at least some subsurface or surface skin, tissue, limb or organ features or lumens as viewed at, from, from within, through or out of a second skin, tissue, limb or organ portion(s) such that the practitioner may then or thereafter one or both of: (i) utilize the feature location information for a patient-beneficial purpose, or (ii) utilize the feature location information to guide or assist a subcutaneous, tissue-subsurface, cutaneous or skin-targeted non-invasive or invasive medical procedure;
the first and second skin, tissue, limb or organ portions being, at least in part, different or the same portions; and
one or more second skin, tissue, limb or organ portions allowing at least one of direct or indirect viewing or recording of the device-produced or excited optical contrast.

2. The device of claim 1 wherein said at least one discrete single or multielement light source includes at least one of an LED source, semiconductor-chip source, OLED source, laser source, surface-emitting laser source, incandescent source, halogen source, fluorescing source, phosphorescent source, plasma source, flashlamp source, electrical discharge source, arc-source, filament source or optical-excitation or agent exciting light source, said powering being by an external and/or internal energy storage source.

3. The device of claim 1 wherein said at least one distributed light source includes or utilizes any of (i) an optically leaky or non-leaky solid or hollow optical waveguide, (ii) an array of discrete single element or multielement light sources, (iii) a glass, ceramic or polymeric-containing optical waveguide of one, two or three dimensions, (iv) an optical fiber or conduit of any type, including solid and hollow-reflective and refractive optical conduits, (v) an optical diffuser, lens, reflector, prism, mirror or grating, including microarrays thereof, (vi) a microlens or lens array or a fiber-optic coupled lens, (vii) any type of physically, mechanically or electro-optically scanned light source, (viii) any type of diffractive optics, reflective optics or gradient-index optics, (ix) any type of optical-excitation or agent-excitation light source, or (x) a bulk light-emitting material or film, including an OLED emitter, said powering being by an external and/or internal energy storage source.

4. The device of claim 1 wherein the device supports the operation of any of: an electrotherapy device, including a TENS device, a tissue ingoing or outgoing electrophoresis or iontophoresis device, a biomedical or physiological sensor for any purpose, whether being implanted or not, a vibratory pain-reduction device, a tissue or feature marking or measuring component, a warming or cooling pain reduction device, a vibratory needle puncture-assistance device, a drug infusion device, a tissue optical transmissivity modification device or a device-guided or device-assisted implement,
wherein by supporting is meant any one or more of:
a) the device provides power therefore;
b) the device provides power connectivity or data connectivity therefore;
c) the device provides mechanical guiding, mounting or measuring surfaces or features therefore;
d) the device dispenses or stores a drug, medicament, medium or gel therefore; or
e) the device provides guidance—for or procedural assistance therefore.

5. The device of claim 1 wherein the optical contrast-improvement means includes at least one of:
a) an optical filter that selectively admits or rejects at least one wavelength of light from any of (i) light entering the skin, tissue, limb or organ for a purpose of optical contrast production or light excitation, or (ii) light falling on the skin, tissue, limb or organ as from ambient light which might interfere with a desired optical contrast, or (iii) returned, reflected, scattered, diffracted, refracted or excited light leaving the skin, tissue, limb or organ and passing to the practitioner's eye along an optical path or to an indirect contrast imaging or recording means providing the practitioner with a visible-contrast image;
b) an optical polarizer that polarizes at least some skin, tissue, limb or organ ingoing or outgoing light of at least one wavelength somewhere on an optical path;
c) a skin, tissue, limb or organ stretching, deformation or deformation-retainment means;
d) a skin, tissue, limb or organ optical-transmissivity improvement means, including one or both of (i) a skin, tissue, limb or organ optical-property manipulation medium that is delivered to one or both of the skin, tissue, limb or organ surface or subsurface, (ii) a means to remove a dead, damaged or diseased surface skin, tissue, limb or organ layer or portion thereof;
e) an ambient light-excluding shield, shroud, cover or room-light on/off or dimmer control, at least some ambient light being intercepted or avoided;
f) delivery or application of an optically-excitable or optically viewable dye, particles(s), nanoparticle(s), surface-coating, bulk-dopant or agent to a surface, subcutaneous or subsurface skin, tissue, limb or organ portion, to an assisted or guided implement or to an implantable implant or sensor, said viewing of excited contrast being at least one of unassisted or assisted;
g) an antireflective film or coating employed anywhere on an actual or potential optical path; or
h) a means to excite or induce optical contrast in any of a natural healthy or unhealthy skin, tissue, limb or organ tissue or body fluid, said excited or induced contrast being human visible or made human visible using a non-visible-to-visible conversion and redisplay or recording approach,

6. A kit containing at least one assistive or guiding device of claim 1 and any one or more of:
a) one or more disposable or non-disposable components used in support of the device or of a device assisted or guided procedure, including possibly splashguards, glove(s), eye-protection, optical filters, lenses, polarizers, antireflection components, ambient light-shrouds, disposable device wraps or membranes, drug patches or wipes, excitable contrast agents, tissue marking components or mediums, TENS or other electrotherapy electrodes, gels, components or materials, magnetic-field therapy components or materials, combination optical-filter/splashguards or needles;
b) a charger, external power supply, battery, power-pack, external powering means or any type of power or data cabling, connectors or jacks;
c) replacement, spare or different color or optical-parameter light sources;
d) an extra or different housing arm member, optionally including different length or shaped arms or arms with different color lights, the housing arm(s) optionally being movable or deformable;
e) a device patient-fixation means optionally including a selection thereof for different patient types and/or sizes and/or an adjustable fixation means;
f) an ultrasonic device utilized to validate or find flowing lumens, optionally including one through which a penetrating needle may be passed into tissue, said ultrasonic device possibly being mounted on a bridging member or element and possibly providing imaging or flow information;
g) a bridging element of any type, disposable or non-disposable;
h) a mating or guiding means used to mate with or guide a procedural implement, optionally including a syringe or biopsy needle, the means not necessarily also being or utilizing a bridging element;
i) a tissue marking or measuring entity, member or marking-medium, including tape or ink used to at least temporarily mark tissue locations, feature locations or device-locations of interest;
j) an adhesive member(s) used to fixate the device, an implement, a sensor, an electrode or a device fixation strap;
k) a tissue-distortion or rolling vein clamping or loading component;
l) an article of disposable or non-disposable device-supportive eyewear or headwear;
m) a selection of types or sizes of device fixation means, optionally including straps and malleable, elastic or viscoelastic limb-clamping or limb enwrapping members;
n) an imaging device or chip, optionally including an infrared or ultraviolet imaging device and optionally lenses or connectors therefore;
o) a room-light dimmer or switch, optionally having a wireless capability;
p) one or more amusing or entertaining light-show programs or audio recordings for amusing, distracting or entertaining patients, optionally stored on a memory media pluggable into the device or into a connected PC or PDA;
q) any accidental needle-stick prevention material, component or member regardless of whether it is mounted to the device or tissue or not or whether it is worn by the practitioner;
r) a display on which to observe optical contrast gathered by an imaging means, at least the displayed contrast being human-visible, whether or not said display is device mountable;
s) any type of supportive cables, connectors, jacks, optical fibers or umbilicals;
t) any type of holder for the device, optionally including a pocket-based holder, a waistband or belt-based holder, a desktop holder, a wall holder, any holder having an integrated recharger or a wrist-leash to prevent droppage;
u) any type of skin-surface abrasion media or member;
v) any type of skin or tissue optical-transmissivity improvement medium;
w) any cable or wireless item for network or PC connectivity, including for display or device powering.
x) any type of consumable or disposable drug, medicament, optical-transmissivity enhancing medium or electrode gel used in combination with the assistive or guiding device
y) instructions for use; or
z) ordering information for consumables and/or device servicing information, preferably internet web based.

7. The device of claim 1 wherein said one or more discrete and/or distributed light sources is, includes or is optically-sourced or fed by any of (i) a surface-mount light source, (ii) a standard 3 mm or 5 mm diameter LED, (iii) any LED or laser industry-standardized-pinout or standardized-package light-emitting device (iv) a human-visible or human-non-visible light emitting light source, (v) an infrared or ultraviolet light source, including any emitting in any portion of any one or more of the NIR, SWIR, MWIR, LWIR, VLWIR or UV wavelength regimes, (vi) an arc, discharge, halogen, plasma or glowing filament-based source, (vii) any diode device, (viii) any laser or lasing device, including solid-state lasers, ion-lasers, dye-lasers, vertical-cavity lasers, excimer-lasers and gaseous lasers, (ix) a light source that excites an excitable agent or tissue/bodyfluid constituent to produce excited light, including those wherein said agent is delivered into the patient tissue or wherein said agent is part of a light-producing device light source, (x) any semiconductor chip based light source, or (xi) a light source known to provide desirable contrast by any mechanism including as by differential transmissivity, opacity, reflectivity or optical-excitation.

8. The device of claim 1 wherein any one or more of:
a) one or more discrete or distributed light sources is operated in a continuous or pulsed manner;
b) a discrete or distributed pulsed light source is operated below, at or above a visual-fusion rate;
c) two or more discrete or distributed light sources are operated in a sequential, interleaved or simultaneous manner;
d) one or more discrete or distributed light sources can be switched or tuned with respect to color;
e) one or more discrete or distributed light sources has a fixed or controlled beam entry angle into tissue or with respect to a lumen;
f) one or more discrete or distributed light sources has a fixed or controlled beam divergence angle;
g) tissue is beneficially deformed for any reason by the device or its fixation means;
h) tissue is deformed into or extruded into or by the device;
i) optical contrast of features is generated using any one or more of backlighting, sidelighting or toplighting of said features;
j) a guided or assisted implement can be seen inside tissue using produced or excited optical contrast;
k) a guided or assisted implement of (j) is treated or capable of emitting light to enhance its visibility;
l) a bridging element is useable with the device, whether permanently or temporarily attached to the device;
m) a light shroud or light-gasket is employed by the device;
n) at least one discrete or distributed light source is or are capable of emitting any one or more of a normally human-visible red, red-orange, orange, orange-yellow, yellow, amber or white color or wavelength(s);
o) at least one discrete or distributed light source is an infrared light source emitting in any of the following wavelength ranges:
or

| | |
|---|---|
| NIR or Near Infrared | approx 0.7-1.0 microns, |
| SWIR or Short Wave Infrared | approx 1.0-3.0 microns, |
| MWIR (or MIR) or Mid Infrared | approx 3.0-8.0 microns, |
| LWIR (or LIR) or Long Wave Infrared | approx 8.0-12.0 microns, |
| VLWIR or Very Long Wave Infrared | approx 12-30 microns; |

p) at least one discrete or distributed light source is an ultraviolet or UV source or emits in the 255 nanometers to 420 nanometers wavelength range;
q) a feature of interest is optically contrasted by two or more light sources from different angles, optionally sequentially;
r) two or more discrete light sources form an array or two or more distributed light sources are employed, or
s) illumination or excitation light is delivered at an angle or angles to features of interest to minimize a shadowing phenomenon, including delivering illumination in planes generally parallel to elongated lumens.

9. A guiding or assistive device for guiding, assisting or implementing pain-free or reduced-pain patient skin, tissue, limb, organ or lumen-disrupting, penetrating or painful medical procedures therein or thereat, the device including:
a) an optical contrast producing means for producing surface-visible or surface-imageable optical contrast of patient features of interest in the skin or in a subsurface tissue, organ, lumen or body-fluid, the contrast producing means utilizing at least some skin or tissue injected or penetrating light deliverable by one or more discrete light sources and/or one or more distributed light sources; and
b) a real-pain or perceived-pain reduction means utilizing at least one TENS, other electrotherapy, magnetic-field based therapy or a drug, heating, cooling or vibratory pain-relief mechanism, the pain reduction mechanism being applied at one or more points in time to reduce some patient real or perceived pain related to a future, current or prior procedure, the procedure carried out with optical assistance or guidance from the assistive device,
the optical contrast being caused by at least one mechanism of optical attenuation, optical scattering, optical excitation, optical reflection, optical refraction, optical diffraction or by any of optical shadowing, optical backlighting, optical side-lighting or optical toplighting, an optical contrast agent or optically excitable agent optionally being employed.

10. The device of claim 9 wherein said at least one discrete single or multielement light source includes at least one of an LED source, semiconductor-chip source, OLED source, laser source, surface-emitting laser source, incandescent source, halogen source, fluorescing source, phosphorescent source, plasma source, flashlamp source, electrical discharge source, arc-source, filament source or optical-excitation or agent exciting light source.

11. The device of claim 9 wherein said at least one distributed light source includes or utilizes any of (i) an optically leaky or non-leaky solid or hollow optical waveguide, (ii) an array of discrete single element or multielement light sources, (iii) a glass, ceramic or polymeric-containing optical waveguide of one, two or three dimensions, (iv) an optical fiber or conduit of any type, including solid and hollow-reflective and refractive optical conduits, (v) an optical diffuser, lens, reflector, prism, mirror or grating, including microarrays thereof, (vi) a microlens or lens array or a fiber-optic coupled lens, (vii) any type of physically, mechanically or electro-optically scanned light source, (viii) any type of diffractive optics, reflective optics or gradient-index optics, (ix) any type of optical-excitation or agent-excitation light source, or (x) a bulk light-emitting material or film, including an OLED emitter.

12. The device of claim 9 wherein the device is attached or juxtaposed to a patient's body, organ, skin, tissue or limb, said attachment optionally still allowing for some device scanning or motion relative to said body, tissue or organ, the attachment or juxtaposition utilizing any one or more of:
a) any type of retainment strap, cord, chain, clamp, belt, suction or adhesive;
b) any type of elastic, plastic, malleable or spring loaded clamping member or arm;
c) the at least partial enwrapping or surrounding of a patient limb or organ by the inherent surrounding shape of the device itself, including for a bracelet or cuff shaped device; or
d) the device is held on the patient by the practitioner or the patient.

13. A method of executing a skin, tissue, organ, limb, body-fluid or lumen related, medically-related or health-related procedure with minimal or no pain, the procedure which might otherwise cause perceived or real pain for a patient or subject, the method utilizing both an optical-contrast provision assistive or guiding device and a device-integrated or device-supporting pain-reduction means comprising:
a) an optical contrast producing or exciting device for viewing or locating skin, tissue, limb, organ, body-fluid or lumen-related subsurface features of interest, said features(s) being the target of the intended procedure or being features to be avoided during said procedure, the device assisting or guiding the procedure, optical light being delivered or excited by at least one discrete light source and/or at least one distributed light source, the optical contrast being caused by at least one mechanism of optical attenuation, optical scattering, optical excitation, optical reflection, optical refraction, optical diffraction or by any of optical shadowing, optical backlighting, optical side-lighting or optical toplighting, an optical contrast agent or optically excitable agent optionally being employed; and
b) at least one of a heating, cooling, vibratory, acoustic, drug-based, TENS-based or other electrotherapy-based or magnetic-field application-based pain reduction means that can be applied in an appropriate timed and locational relationship to the actual site of the procedure and/or site of the procedural pain, wherein:
i) the pain reducing means is operated or applied at least one of before, during or after the execution of the procedure in support of a pain-limited or pain-free procedure wherein real or perceived pain relief is delivered;
ii) the assistive or guiding device is optionally fixated, loosely and/or snugly, to a limb, tissue, organ or skin of interest at least one point in time in support of one or more of feature-finding, tissue or lumen manipulation or procedure delivery; and
iii) the assistive or guiding device optionally provides operational support for, if not also actually copackages or physically supports, one or more of said pain-reduction means.

14. The method of claim 13 wherein said at least one discrete single or multielement light source includes at least one of an LED source, semiconductor-chip source, OLED source, laser source, surface-emitting laser source, incandescent source, halogen source, fluorescing source, phosphorescent source, plasma source, flashlamp source, electrical discharge source, arc-source, filament source or optical-excitation or agent exciting light source.

15. The method of claim 13 wherein said at least one distributed light source includes or utilizes any of (i) an optically leaky or non-leaky solid or hollow optical waveguide, (ii) an array of discrete single element or multielement light sources, (iii) a glass, ceramic or polymeric-containing optical waveguide of one, two or three dimensions, (iv) an optical fiber or conduit of any type, including solid and hollow-reflective and refractive optical conduits, (v) an optical diffuser, lens, reflector, prism, mirror or grating, including microarrays thereof, (vi) a microlens or lens array or a fiber-optic coupled lens, (vii) any type of physically, mechanically or electro-optically scanned light source, (viii) any type of diffractive optics, reflective optics or gradient-index optics, (ix) any type of optical-excitation or agent-excitation light source, or (x) a bulk light-emitting material or film, including an OLED emitter.

16. The method of claim 13 wherein the device is attached or juxtaposed to a patient's body, organ, skin, tissue or limb, said attachment optionally still allowing for some device or device-portion scanning or motion relative to said body, tissue or organ, the attachment or juxtaposition utilizing any one or more of:
a) any type of retainment strap, cord, chain, clamp, belt, suction or adhesive;
b) any type of elastic, plastic, malleable or spring loaded clamping member or arm; or
c) the at least partial enwrapping or surrounding of a patient limb or organ by the inherent surrounding shape of the device itself, including for a bracelet or cuff shaped device.

17. A method of favorably dilating a lumen in preparation or support of a medical procedure, including a needle-stick, comprising:
a) optically determining a skin or tissue region containing or overlying said lumen utilizing at least one discrete light source and/or at least one distributed light source mounted in or on a light-housing juxtaposable, presentable-to or mountable to a patients tissues, the light housing including at least one discrete and/or distributed light source, optical contrast being caused by at least one mechanism of optical attenuation, optical scattering, optical excitation, optical reflection, optical refraction, optical diffraction or by any of optical shadowing, optical backlighting, optical side-lighting or optical toplighting, an optical contrast agent or optically excitable agent optionally being employed; and
b) applying TENS or other electrotherapy excitation, vibratory or acoustic excitations to said lumen region sufficient to cause the desired dilation of the lumen for a useful period, the exciter(s) itself not necessarily being located directly over or adjacent said lumen but at least its excitations being coupled into said lumen region.

18. The device of claim 17 wherein said at least one discrete single or multielement light source includes at least one of an LED source, semiconductor-chip source, OLED source, laser source, surface-emitting laser source, incandescent source, halogen source, fluorescing source, phosphorescent source, plasma source, flashlamp source, electrical discharge source, arc-source, filament source or optical-excitation or agent exciting light source.

19. The device of claim 17 wherein said at least one distributed light source includes or utilizes any of (i) an optically leaky or non-leaky solid or hollow optical waveguide, (ii) an array of discrete single element or multielement light sources, (iii) a glass, ceramic or polymeric-containing optical waveguide of one, two or three dimensions, (iv) an optical fiber or conduit any type, including solid and hollow-reflective and refractive optical conduits, (v) an optical diffuser, lens, reflector, prism, mirror or grating, including microarrays thereof, (vi) a microlens or lens array or a fiber-optic coupled lens, (vii) any type of physically, mechanically or electro-optically scanned light source, (viii) any type of diffractive optics, reflective optics or gradient-index optics, (ix) any type of optical-excitation or agent-excitation light source, or (x) a bulk light-emitting material or film, including an OLED emitter.

20. The device of claim 17 wherein the device is attached or juxtaposed to a patient's body, organ, skin, tissue or limb, said attachment optionally still allowing for some device or device-portion scanning or motion relative to said body, tissue or organ, the attachment or juxtaposition utilizing any one or more of:
a) any type of retainment strap, cord, chain, clamp, belt, suction or adhesive;
b any type of elastic, plastic, malleable or spring loaded clamping member or arm; or
c) the at least partial enwrapping or surrounding of a patient limb or organ by the inherent surrounding shape of the device itself, including for a bracelet or cuff shaped device.

21. The device of claim 17 wherein said lumen-region dilation excitations are produced in or on the device or are patient or practitioner-applied utilizing any of power, logic circuitry, physical or wireless connectivity, mechanical guidance, electrodes, fixation or physical alignment provided by the device.

22. A cuff-like optical assistance or guidance device for application to medical procedures, exams, treatments, therapies or surgeries comprising:
a) a cuff-like entity that wraps around or surrounds at least a portion of a human limb or organ, said wrapping or surrounding being at least in part one or both of in contact with said limb or being proximal or gapped from or to said limb surface;
b) the cuff-like entity including one or more single element or multielement discrete light sources and/or one or more distributed light sources capable of injecting light into or upon a first skin or tissue portion; and
c) the cuff-like entity including or defining a second skin or tissue portion wherefrom or whereat optical contrast of tissue surface or subsurface features produced by said injected light may be observed in at least one of an unaided or aided manner by a practitioner,
wherein:
the optical contrast being caused by at least one mechanism of optical attenuation, optical scattering, optical excitation, optical reflection, optical refraction, optical diffraction or by any of optical shadowing, optical backlighting, optical side-lighting or optical toplighting, an optical contrast agent or optically excitable agent optionally being employed; and
the device allowing for any required access of a device-guided or assisted procedural implement to the patient's tissues.

23. The device of claim 22 wherein said at least one discrete single or multielement light source includes at least one LED source, semiconductor-chip source, OLED source, laser source, surface-emitting laser source, incandescent source, halogen source, fluorescing source, phosphorescent source, plasma source, flashlamp source, electrical discharge source, arc-source, filament source or optical-excitation or agent exciting light source.

24. The device of claim 22 wherein said at least one distributed light source includes or utilizes any of (i) an optically leaky or non-leaky solid or hollow optical waveguide, (ii) an array of discrete single element or multielement light sources (iii) a glass, ceramic or polymeric-containing optical waveguide of one, two or three dimensions, (iv) an optical fiber or conduit any type, including solid and hollow-reflective and refractive optical conduits, (v) an optical diffuser, lens, reflector, prism, mirror or grating, including microarrays thereof, (vi) a microlens or lens array or a fiber-optic coupled lens, (vii) any type of physically, mechanically or electro-optically scanned light source, (viii) any type of diffractive optics, reflective optics or gradient-index optics, (ix) any type of optical-excitation or agent-excitation light source, or (x) a bulk light emitting material or film, including an OLED emitter.

25. The device of claim 22 wherein the device any one or more of: a) can be attached or mounted to one or more of a patient's body, limb, tissue or organ, b) utilizes for optical contrast improvement any of a filter, antireflection coating, polarizer. optical contrast agent, light shroud or ambient-light switching or modulation element, c) utilizes for safety any of a blood-splash protection feature, a disposable polymeric device membrane, or a shroud or shield, d) utilizes, records or presents an image or display of optically contrasted features, e) is fittable or shapeable to a particular patient, limb or organ using an exchangeable, adjustable, malleable, elastic, spring-loaded or size-settable feature or member, f) utilizes a disposable component, including a disposable optical filter that might double as a splash guard, g) has a practitioner self needle-stick protection feature, h) has a practitioner useable finger-rest or finger guidance feature, i) has a practitioner useable guiding or mechanical reference feature for supporting a guided or assisted implement, including a syringe or biopsy needle, j) dispenses or delivers a drug for any purpose, optionally including lumen dilation encouragement or pain-reduction, or k) treats real, anticipated or perceived pain with an acoustic, vibratory, electrotherapeutic, heating, cooling or magnetic component.
